SEQUENCE LISTING (21 sequences)

SEQ ID NO: 1 - protein, 345 aa, Oryza sativa
MGRSPCCEKEGLKKGPWTPEEDQKLLAYIEQHGHGCWRSLPSKAGLQRCGKSCRLRWTNYLRPDIKRGKFSLQEEQTIIQLHALLGNRWSAIATHLPKRTDNEIKNYWNTHLKKRLAKMGIDPVTHKPRSDVAGAGGGGGGAAGGAAGAQHAKAAAHLSHTAQWESARLEAEARLAREAKLRALAASATPGAPHLPAPPASAAAAAAAHGLDSPTSTLSFSESAVLATVLEAHGAAAAAAARAAMQPMQAYDEACKDQHWGDVDAADVGFPGAGAGFTGLLLEGSLNQIPRPAGRDAEADGEFQETEEEKNYWNSILNLVNSSSAPMSTAVVVPASHAYSPAPDF

SEQ ID NO: 2 - DNA, 1038 bp, Oryza sativa
atggggcgatcgccgtgctgcgagaaggaggggctcaagaaggggccatggacgccggaggaggaccagaagctgctggcctacatcgagcagcacggccacggctgctggcgctcgctaccctccaaggccgggctgcagcggtgcggcaagagctgccgactccggtggacgaactacctccggccggacatcaagaggggcaagttcagcctgcaggaggagcagaccatcatccagctccacgcccttctcggcaacaggtggtcggcgatcgcgacgcacctgccgaagcgcacggacaacgagatcaagaactactggaacacgcacctaaagaagcggctggccaagatggggatcgacccggtcacgcacaagccgcgctccgacgtggccggcgcgggcggcggcggcggaggtgcggccggcggcgcggcgggcgcgcagcacgccaaggccgcggcgcacctcagccacacggcgcagtgggagagcgcgcggctcgaggcggaggcgcgcttggctcgggaggccaagctgcgcgcgctcgcggcctccgcgaccccgggcgcgccgcacctcccggcaccccccgcgtcggcggcggcggcggcggcggctcacggcctcgactcgccgacgtccacgctgagcttctcggagagcgcggtgctcgccaccgtgctggaggcgcacggcgccgccgccgcggcggccgcgcgcgccgccatgcagcccatgcaggcgtacgacgaggcgtgcaaggaccagcactggggcgacgtcgacgccgccgacgtgggcttccccggcgccggagcggggttcacgggcctactcctcgaaggctccttgaaccagatcccgaggccggcggggagagacgcggaagccgacggcgagttccaggagaccgaggaggagaagaactactggaacagcatactgaacctggtgaactcctcgtcggcacctatgtcgacggccgtggttgtgcccgcctcccacgcgtactcgccggcgccggacttctga

SEQ ID NO: 3 - DNA, 2623 bp, Oryza sativa
ggcctctctctctctctctctctctctcacacacacacacactctcactgactctgctgctgcattagtcactcgcagagagccacagctccctgcaaagaagatctctcgtagtgaattgcctcgatcacgtactactacacatagacctactacttgagcccgagagaagaggagaggaggaagaaccagagggcgtcgaagatcatcggggaggagttttcctagagctcgctgctgctgttgcttttctccggcgatggggcgatcgccgtgctgcgagaaggaggggctcaagaaggggccatggacgccggaggaggaccagaagctgctggcctacatcgagcagcacggccacggctgctggcgctcgctaccctccaaggccggtaagcgccgcttatctagcttaaatttcttctcaacctctgcaatcctagctgcaatgttcggtcgaggcgatcgatcctcgaggctggctactctctgaactctgatctgaggtgcatgcaaaccgtgacaatcgtgtgcagggctgcagcggtgcggcaagagctgccgactccggtggacgaactacctccggccggacatcaagaggggcaagttcagcctgcaggaggagcagaccatcatccagctccacgcccttctcggcaacaggtgatcgattacttcgttttcgcatggatgcatcatgcatacaagatacgtagtgcacaactctccctcctgctagctgctcgctcgttcttcacctcgcacccggagcacatttaattccgtaatcgcgatggaacccttgattctcctgcacgaattttgactgctagtacttgttgctgaccggcaggtcaagaacacactagccagtagccaccattctgcaccgtagtcttggcagacatttatgaaagggttatgcaatgcaagggttggaacacggagcttagccaggggatgtgtaaatttcgcaggccgtgctacttacttgctgtccccgtacacacctgcttcagcattttgtccgtaacaaaccgtactgtccatagattaacacacaagctaggctaaaaattcttacgttagaacagaatcatcacttgttttcgttttgttcacacgtaatgctgcattgctcatcttttgcccgtcgaacaaccacgcattagctgtgagcacagaccaatcaatgcatgcatcaacaagggaaaaagtgtgaaaaggttgggcagtgagaggctcggcccagaattttcctttcttttctcccatatgattcggcattcaagctcgtcatttaaggaggcgagcccccccatcattgtggaccaaaactggggtttggtccactgttgccacctgcccctcttcccattttgactcacagcttccgatcatctctgccctctgtctgtactacgccacgcacgccttaaatcacaccgccgattatttacgttttcaagagtgctgtttgtttaattttgtcactgcgaatggagggcttttgacgtgcgattttcctgatcttttttcttggccggcgttggcgttgttgttgttttgcaggtggtcggcgatcgcgacgcacctgccgaagcgcacggacaacgagatcaagaactactggaacacgcacctaaagaagcggctggccaagatggggatcgacccggtcacgcacaagccgcgctccgacgtggccggcgcgggcggcggcggcggaggtgcggccggcggcgcggcgggcgcgcagcacgccaaggccgcggcgcacctcagccacacggcgcagtgggagagcgcgcggctcgaggcggaggcgcgcttggctcgggaggccaagctgcgcgcgctcgcggcctccgcgaccccgggcgcgccgcacctcccggcaccccccgcgtcggcggcggcggcggcggcggctcacggcctcgactcgccgacgtccacgctgagcttctcggagagcgcggtgctcgccaccgtgctggaggcgcacggcgccgccgccgcggcggccgcgcgcgccgccatgcagcccatgcaggcgtacgacgaggcgtgcaaggaccagcactggggcgacgtcgacgccgccgacgtgggcttccccggcgccggagcggggttcacgggcctactcctcgaaggctccttgaaccagatcccgaggccggcggggagagacgcggaagccgacggcgagttccaggagaccgaggaggagaagaactactggaacagcatactgaacctggtgaactcctcgtcggcacctatgtcgacggccgtggttgtgcccgcctcccacgcgtactcgccggcgccggacttctgagcggagaaaacctcgccggcatcaaacttgattcgatctcatgacacagtaaatagtttagcaatgattgtcgaataagatgggactaatattaatgttagtaattattaatcacgttcttgggttaacctgacaatgctttcgattaaacttgtgggcaagaacttcaactgttcaaggctgtatcgacatttgaaattcgatgcttgttttgttcggtgttcttatagaatgtgtaaaacactgaaactactatcagagaatgtagcatcc

SEQ ID NO: 4 - DNA, 72 bp, Artificial Sequence
  Synthesized
tagagctagaaatagcaagttaaaataaggctagtccgttatcaacttgaaaaagtggcaccgagtcggtgc

SEQ ID NO: 5 - DNA, 533 bp, Artificial Sequence
  Synthesized
ctcgcactggaatcggcagcaaaggaaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgtgatgtgaaggagtaagataaacttaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcaccaaggccggtaagcgccgcgttttagagctagaaatagcaagttaaaataaggctagtccgttatcaacttgaaaaagtggcaccgagtcggtgctttttttcaagagcttggagtggatggacc

SEQ ID NO: 6 - protein, 359 aa, Artificial Sequence
  Synthesized
MGRSPCCEKEGLKKGPWTPEEDQKLLAYIEQHGHGCWRSLPSSRAAAVRQELPTPVDELPPAGHQEGQVQPAGGADHHPAPRPSRQQVVGDRDAPAEAHGQRDQELLEHAPKEAAGQDGDRPGHAQAALRRGRRGRRRRRCGRRRGGRAARQGRGAPQPHGAVGERAARGGGALGSGGQAARARGLRDPGRAAPPGTPRVGGGGGGGSRPRLADVHAELLGERGARHRAGGARRRRRGGRARRHAAHAGVRRGVQGPALGRRRRRRRGLPRRRSGVHGPTPRRLLEPDPEAGGERRGSRRRVPGDRGGEELLEQHTEPGELLVGTYVDGRGCARLPRVLAGAGLLSGENLAGIKLDSIS

SEQ ID NO: 7 - DNA, 1080 bp, Artificial Sequence
  Synthesized
atggggcgatcgccgtgctgcgagaaggaggggctcaagaaggggccatggacgccggaggaggaccagaagctgctggcctacatcgagcagcacggccacggctgctggcgctcgctaccctccagccgggctgcagcggtgcggcaagagctgccgactccggtggacgaactacctccggccggacatcaagaggggcaagttcagcctgcaggaggagcagaccatcatccagctccacgcccttctcggcaacaggtggtcggcgatcgcgacgcacctgccgaagcgcacggacaacgagatcaagaactactggaacacgcacctaaagaagcggctggccaagatggggatcgacccggtcacgcacaagccgcgctccgacgtggccggcgcgggcggcggcggcggaggtgcggccggcggcgcggcgggcgcgcagcacgccaaggccgcggcgcacctcagccacacggcgcagtgggagagcgcgcggctcgaggcggaggcgcgcttggctcgggaggccaagctgcgcgcgctcgcggcctccgcgaccccgggcgcgccgcacctcccggcaccccccgcgtcggcggcggcggcggcggcggctcacggcctcgactcgccgacgtccacgctgagcttctcggagagcgcggtgctcgccaccgtgctggaggcgcacggcgccgccgccgcggcggccgcgcgcgccgccatgcagcccatgcaggcgtacgacgaggcgtgcaaggaccagcactggggcgacgtcgacgccgccgacgtgggcttccccggcgccggagcggggttcacgggcctactcctcgaaggctccttgaaccagatcccgaggccggcggggagagacgcggaagccgacggcgagttccaggagaccgaggaggagaagaactactggaacagcatactgaacctggtgaactcctcgtcggcacctatgtcgacggccgtggttgtgcccgcctcccacgcgtactcgccggcgccggacttctgagcggagaaaacctcgccggcatcaaacttgattcgatctcatga

SEQ ID NO: 8 - DNA, 2621 bp, Artificial Sequence
  Synthesized
ggcctctctctctctctctctctctctcacacacacacacactctcactgactctgctgctgcattagtcactcgcagagagccacagctccctgcaaagaagatctctcgtagtgaattgcctcgatcacgtactactacacatagacctactacttgagcccgagagaagaggagaggaggaagaaccagagggcgtcgaagatcatcggggaggagttttcctagagctcgctgctgctgttgcttttctccggcgatggggcgatcgccgtgctgcgagaaggaggggctcaagaaggggccatggacgccggaggaggaccagaagctgctggcctacatcgagcagcacggccacggctgctggcgctcgctaccctccagccggtaagcgccgcttatctagcttaaatttcttctcaacctctgcaatcctagctgcaatgttcggtcgaggcgatcgatcctcgaggctggctactctctgaactctgatctgaggtgcatgcaaaccgtgacaatcgtgtgcagggctgcagcggtgcggcaagagctgccgactccggtggacgaactacctccggccggacatcaagaggggcaagttcagcctgcaggaggagcagaccatcatccagctccacgcccttctcggcaacaggtgatcgattacttcgttttcgcatggatgcatcatgcatacaagatacgtagtgcacaactctccctcctgctagctgctcgctcgttcttcacctcgcacccggagcacatttaattccgtaatcgcgatggaacccttgattctcctgcacgaattttgactgctagtacttgttgctgaccggcaggtcaagaacacactagccagtagccaccattctgcaccgtagtcttggcagacatttatgaaagggttatgcaatgcaagggttggaacacggagcttagccaggggatgtgtaaatttcgcaggccgtgctacttacttgctgtccccgtacacacctgcttcagcattttgtccgtaacaaaccgtactgtccatagattaacacacaagctaggctaaaaattcttacgttagaacagaatcatcacttgttttcgttttgttcacacgtaatgctgcattgctcatcttttgcccgtcgaacaaccacgcattagctgtgagcacagaccaatcaatgcatgcatcaacaagggaaaaagtgtgaaaaggttgggcagtgagaggctcggcccagaattttcctttcttttctcccatatgattcggcattcaagctcgtcatttaaggaggcgagcccccccatcattgtggaccaaaactggggtttggtccactgttgccacctgcccctcttcccattttgactcacagcttccgatcatctctgccctctgtctgtactacgccacgcacgccttaaatcacaccgccgattatttacgttttcaagagtgctgtttgtttaattttgtcactgcgaatggagggcttttgacgtgcgattttcctgatcttttttcttggccggcgttggcgttgttgttgttttgcaggtggtcggcgatcgcgacgcacctgccgaagcgcacggacaacgagatcaagaactactggaacacgcacctaaagaagcggctggccaagatggggatcgacccggtcacgcacaagccgcgctccgacgtggccggcgcgggcggcggcggcggaggtgcggccggcggcgcggcgggcgcgcagcacgccaaggccgcggcgcacctcagccacacggcgcagtgggagagcgcgcggctcgaggcggaggcgcgcttggctcgggaggccaagctgcgcgcgctcgcggcctccgcgaccccgggcgcgccgcacctcccggcaccccccgcgtcggcggcggcggcggcggcggctcacggcctcgactcgccgacgtccacgctgagcttctcggagagcgcggtgctcgccaccgtgctggaggcgcacggcgccgccgccgcggcggccgcgcgcgccgccatgcagcccatgcaggcgtacgacgaggcgtgcaaggaccagcactggggcgacgtcgacgccgccgacgtgggcttccccggcgccggagcggggttcacgggcctactcctcgaaggctccttgaaccagatcccgaggccggcggggagagacgcggaagccgacggcgagttccaggagaccgaggaggagaagaactactggaacagcatactgaacctggtgaactcctcgtcggcacctatgtcgacggccgtggttgtgcccgcctcccacgcgtactcgccggcgccggacttctgagcggagaaaacctcgccggcatcaaacttgattcgatctcatgacacagtaaatagtttagcaatgattgtcgaataagatgggactaatattaatgttagtaattattaatcacgttcttgggttaacctgacaatgctttcgattaaacttgtgggcaagaacttcaactgttcaaggctgtatcgacatttgaaattcgatgcttgttttgttcggtgttcttatagaatgtgtaaaacactgaaactactatcagagaatgtagcatcc

SEQ ID NO: 9 - protein, 111 aa, Artificial Sequence
  Synthesized
MGRSPCCEKEGLKKGPWTPEEDQKLLAYIEQHGHGCWRSLPSRPGCSGAARAADSGGRTTSGRTSRGASSACRRSRPSSSSTPFSATGGRRSRRTCRSARTTRSRTTGTRT

SEQ ID NO: 10 - DNA, 336 bp, Artificial Sequence
  Synthesized
atggggcgatcgccgtgctgcgagaaggaggggctcaagaaggggccatggacgccggaggaggaccagaagctgctggcctacatcgagcagcacggccacggctgctggcgctcgctaccctccaggccgggctgcagcggtgcggcaagagctgccgactccggtggacgaactacctccggccggacatcaagaggggcaagttcagcctgcaggaggagcagaccatcatccagctccacgcccttctcggcaacaggtggtcggcgatcgcgacgcacctgccgaagcgcacggacaacgagatcaagaactactggaacacgcacctaa

SEQ ID NO: 11 - DNA, 2622 bp, Artificial Sequence
  Synthesized
ggcctctctctctctctctctctctctcacacacacacacactctcactgactctgctgctgcattagtcactcgcagagagccacagctccctgcaaagaagatctctcgtagtgaattgcctcgatcacgtactactacacatagacctactacttgagcccgagagaagaggagaggaggaagaaccagagggcgtcgaagatcatcggggaggagttttcctagagctcgctgctgctgttgcttttctccggcgatggggcgatcgccgtgctgcgagaaggaggggctcaagaaggggccatggacgccggaggaggaccagaagctgctggcctacatcgagcagcacggccacggctgctggcgctcgctaccctccaggccggtaagcgccgcttatctagcttaaatttcttctcaacctctgcaatcctagctgcaatgttcggtcgaggcgatcgatcctcgaggctggctactctctgaactctgatctgaggtgcatgcaaaccgtgacaatcgtgtgcagggctgcagcggtgcggcaagagctgccgactccggtggacgaactacctccggccggacatcaagaggggcaagttcagcctgcaggaggagcagaccatcatccagctccacgcccttctcggcaacaggtgatcgattacttcgttttcgcatggatgcatcatgcatacaagatacgtagtgcacaactctccctcctgctagctgctcgctcgttcttcacctcgcacccggagcacatttaattccgtaatcgcgatggaacccttgattctcctgcacgaattttgactgctagtacttgttgctgaccggcaggtcaagaacacactagccagtagccaccattctgcaccgtagtcttggcagacatttatgaaagggttatgcaatgcaagggttggaacacggagcttagccaggggatgtgtaaatttcgcaggccgtgctacttacttgctgtccccgtacacacctgcttcagcattttgtccgtaacaaaccgtactgtccatagattaacacacaagctaggctaaaaattcttacgttagaacagaatcatcacttgttttcgttttgttcacacgtaatgctgcattgctcatcttttgcccgtcgaacaaccacgcattagctgtgagcacagaccaatcaatgcatgcatcaacaagggaaaaagtgtgaaaaggttgggcagtgagaggctcggcccagaattttcctttcttttctcccatatgattcggcattcaagctcgtcatttaaggaggcgagcccccccatcattgtggaccaaaactggggtttggtccactgttgccacctgcccctcttcccattttgactcacagcttccgatcatctctgccctctgtctgtactacgccacgcacgccttaaatcacaccgccgattatttacgttttcaagagtgctgtttgtttaattttgtcactgcgaatggagggcttttgacgtgcgattttcctgatcttttttcttggccggcgttggcgttgttgttgttttgcaggtggtcggcgatcgcgacgcacctgccgaagcgcacggacaacgagatcaagaactactggaacacgcacctaaagaagcggctggccaagatggggatcgacccggtcacgcacaagccgcgctccgacgtggccggcgcgggcggcggcggcggaggtgcggccggcggcgcggcgggcgcgcagcacgccaaggccgcggcgcacctcagccacacggcgcagtgggagagcgcgcggctcgaggcggaggcgcgcttggctcgggaggccaagctgcgcgcgctcgcggcctccgcgaccccgggcgcgccgcacctcccggcaccccccgcgtcggcggcggcggcggcggcggctcacggcctcgactcgccgacgtccacgctgagcttctcggagagcgcggtgctcgccaccgtgctggaggcgcacggcgccgccgccgcggcggccgcgcgcgccgccatgcagcccatgcaggcgtacgacgaggcgtgcaaggaccagcactggggcgacgtcgacgccgccgacgtgggcttccccggcgccggagcggggttcacgggcctactcctcgaaggctccttgaaccagatcccgaggccggcggggagagacgcggaagccgacggcgagttccaggagaccgaggaggagaagaactactggaacagcatactgaacctggtgaactcctcgtcggcacctatgtcgacggccgtggttgtgcccgcctcccacgcgtactcgccggcgccggacttctgagcggagaaaacctcgccggcatcaaacttgattcgatctcatgacacagtaaatagtttagcaatgattgtcgaataagatgggactaatattaatgttagtaattattaatcacgttcttgggttaacctgacaatgctttcgattaaacttgtgggcaagaacttcaactgttcaaggctgtatcgacatttgaaattcgatgcttgttttgttcggtgttcttatagaatgtgtaaaacactgaaactactatcagagaatgtagcatcc

SEQ ID NO: 12 - protein, 360 aa, Artificial Sequence
  Synthesized
MGRSPCCEKEGLKKGPWTPEEDQKLLAYIEQHGHGCWRSLPSKGRAAAVRQELPTPVDELPPAGHQEGQVQPAGGADHHPAPRPSRQQVVGDRDAPAEAHGQRDQELLEHAPKEAAGQDGDRPGHAQAALRRGRRGRRRRRCGRRRGGRAARQGRGAPQPHGAVGERAARGGGALGSGGQAARARGLRDPGRAAPPGTPRVGGGGGGGSRPRLADVHAELLGERGARHRAGGARRRRRGGRARRHAAHAGVRRGVQGPALGRRRRRRRGLPRRRSGVHGPTPRRLLEPDPEAGGERRGSRRRVPGDRGGEELLEQHTEPGELLVGTYVDGRGCARLPRVLAGAGLLSGENLAGIKLDSIS

SEQ ID NO: 13 - DNA, 1083 bp, Artificial Sequence
  Synthesized
atggggcgatcgccgtgctgcgagaaggaggggctcaagaaggggccatggacgccggaggaggaccagaagctgctggcctacatcgagcagcacggccacggctgctggcgctcgctaccctccaaaggccgggctgcagcggtgcggcaagagctgccgactccggtggacgaactacctccggccggacatcaagaggggcaagttcagcctgcaggaggagcagaccatcatccagctccacgcccttctcggcaacaggtggtcggcgatcgcgacgcacctgccgaagcgcacggacaacgagatcaagaactactggaacacgcacctaaagaagcggctggccaagatggggatcgacccggtcacgcacaagccgcgctccgacgtggccggcgcgggcggcggcggcggaggtgcggccggcggcgcggcgggcgcgcagcacgccaaggccgcggcgcacctcagccacacggcgcagtgggagagcgcgcggctcgaggcggaggcgcgcttggctcgggaggccaagctgcgcgcgctcgcggcctccgcgaccccgggcgcgccgcacctcccggcaccccccgcgtcggcggcggcggcggcggcggctcacggcctcgactcgccgacgtccacgctgagcttctcggagagcgcggtgctcgccaccgtgctggaggcgcacggcgccgccgccgcggcggccgcgcgcgccgccatgcagcccatgcaggcgtacgacgaggcgtgcaaggaccagcactggggcgacgtcgacgccgccgacgtgggcttccccggcgccggagcggggttcacgggcctactcctcgaaggctccttgaaccagatcccgaggccggcggggagagacgcggaagccgacggcgagttccaggagaccgaggaggagaagaactactggaacagcatactgaacctggtgaactcctcgtcggcacctatgtcgacggccgtggttgtgcccgcctcccacgcgtactcgccggcgccggacttctgagcggagaaaacctcgccggcatcaaacttgattcgatctcatga

SEQ ID NO: 14 - DNA, 2624 bp, Artificial Sequence
  Synthesized
ggcctctctctctctctctctctctctcacacacacacacactctcactgactctgctgctgcattagtcactcgcagagagccacagctccctgcaaagaagatctctcgtagtgaattgcctcgatcacgtactactacacatagacctactacttgagcccgagagaagaggagaggaggaagaaccagagggcgtcgaagatcatcggggaggagttttcctagagctcgctgctgctgttgcttttctccggcgatggggcgatcgccgtgctgcgagaaggaggggctcaagaaggggccatggacgccggaggaggaccagaagctgctggcctacatcgagcagcacggccacggctgctggcgctcgctaccctccaaaggccggtaagcgccgcttatctagcttaaatttcttctcaacctctgcaatcctagctgcaatgttcggtcgaggcgatcgatcctcgaggctggctactctctgaactctgatctgaggtgcatgcaaaccgtgacaatcgtgtgcagggctgcagcggtgcggcaagagctgccgactccggtggacgaactacctccggccggacatcaagaggggcaagttcagcctgcaggaggagcagaccatcatccagctccacgcccttctcggcaacaggtgatcgattacttcgttttcgcatggatgcatcatgcatacaagatacgtagtgcacaactctccctcctgctagctgctcgctcgttcttcacctcgcacccggagcacatttaattccgtaatcgcgatggaacccttgattctcctgcacgaattttgactgctagtacttgttgctgaccggcaggtcaagaacacactagccagtagccaccattctgcaccgtagtcttggcagacatttatgaaagggttatgcaatgcaagggttggaacacggagcttagccaggggatgtgtaaatttcgcaggccgtgctacttacttgctgtccccgtacacacctgcttcagcattttgtccgtaacaaaccgtactgtccatagattaacacacaagctaggctaaaaattcttacgttagaacagaatcatcacttgttttcgttttgttcacacgtaatgctgcattgctcatcttttgcccgtcgaacaaccacgcattagctgtgagcacagaccaatcaatgcatgcatcaacaagggaaaaagtgtgaaaaggttgggcagtgagaggctcggcccagaattttcctttcttttctcccatatgattcggcattcaagctcgtcatttaaggaggcgagcccccccatcattgtggaccaaaactggggtttggtccactgttgccacctgcccctcttcccattttgactcacagcttccgatcatctctgccctctgtctgtactacgccacgcacgccttaaatcacaccgccgattatttacgttttcaagagtgctgtttgtttaattttgtcactgcgaatggagggcttttgacgtgcgattttcctgatcttttttcttggccggcgttggcgttgttgttgttttgcaggtggtcggcgatcgcgacgcacctgccgaagcgcacggacaacgagatcaagaactactggaacacgcacctaaagaagcggctggccaagatggggatcgacccggtcacgcacaagccgcgctccgacgtggccggcgcgggcggcggcggcggaggtgcggccggcggcgcggcgggcgcgcagcacgccaaggccgcggcgcacctcagccacacggcgcagtgggagagcgcgcggctcgaggcggaggcgcgcttggctcgggaggccaagctgcgcgcgctcgcggcctccgcgaccccgggcgcgccgcacctcccggcaccccccgcgtcggcggcggcggcggcggcggctcacggcctcgactcgccgacgtccacgctgagcttctcggagagcgcggtgctcgccaccgtgctggaggcgcacggcgccgccgccgcggcggccgcgcgcgccgccatgcagcccatgcaggcgtacgacgaggcgtgcaaggaccagcactggggcgacgtcgacgccgccgacgtgggcttccccggcgccggagcggggttcacgggcctactcctcgaaggctccttgaaccagatcccgaggccggcggggagagacgcggaagccgacggcgagttccaggagaccgaggaggagaagaactactggaacagcatactgaacctggtgaactcctcgtcggcacctatgtcgacggccgtggttgtgcccgcctcccacgcgtactcgccggcgccggacttctgagcggagaaaacctcgccggcatcaaacttgattcgatctcatgacacagtaaatagtttagcaatgattgtcgaataagatgggactaatattaatgttagtaattattaatcacgttcttgggttaacctgacaatgctttcgattaaacttgtgggcaagaacttcaactgttcaaggctgtatcgacatttgaaattcgatgcttgttttgttcggtgttcttatagaatgtgtaaaacactgaaactactatcagagaatgtagcatcc

SEQ ID NO: 15 - DNA, 20 bp, Artificial Sequence
  Synthesized
ccaaggccggtaagcgccgc

SEQ ID NO: 16 - DNA, 24 bp, Artificial Sequence
  Synthesized
ggcaccaaggccggtaagcgccgc

SEQ ID NO: 17 - DNA, 24 bp, Artificial Sequence
  Synthesized
aaacgcggcgcttaccggccttgg

SEQ ID NO: 18 - DNA, 38 bp, Artificial Sequence
  Synthesized
ttcagaggtctctctcgcactggaatcggcagcaaagg

SEQ ID NO: 19 - DNA, 37 bp, Artificial Sequence
  Synthesized
agcgtgggtctcgaccgggtccatccactccaagctc

SEQ ID NO: 20 - DNA, 23 bp, Artificial Sequence
  Synthesized
cccgacatagatgcaataacttc

SEQ ID NO: 21 - DNA, 22 bp, Artificial Sequence
  Synthesized
gcgcggtgtcatctatgttact

(19) United States
(12) Patent Application Publication
(10) Pub. No.: US 2023/0151383 A1
(43) Pub. Date: May 18, 2023

(54) METHOD FOR PREPARING RICE PHOTOSENSITIVE MALE STERILE MATERIAL AND RELATED GENES THEREOF

(71) Applicant: HUNAN HYBRID RICE RESEARCH CENTER, Changsha (CN)

(72) Inventors: Li LI, Changsha (CN); Mudan QIU, Changsha (CN); Yixing LI, Changsha (CN); Dabing ZHANG, Changsha (CN); Shufeng SONG, Changsha (CN); Tiankang WANG, Changsha (CN)

(21) Appl. No.: 17/997,117

(22) PCT Filed: Apr. 26, 2021
(86) PCT No.: PCT/CN2021/089796

§ 371 Date: Jan. 20, 2023

(30) Foreign Application Priority Data
Apr. 27, 2020 (CN) 202010342657.1

(51) Int. Cl. C12N 15/82; C07K 14/415

(52) U.S. Cl. CPC C12N 15/8289; C12N 15/8213; C07K 14/415

(57) ABSTRACT

The present invention discloses a method for preparing a rice photosensitive male sterile material and related genes thereof. The method for preparing photosensitive male sterile rice of the present invention includes: reducing the abundance of protein RMS1 in the target rice, reducing the activity of the protein RMS1 in the target rice or reducing the content of the protein RMS1 in the target rice to obtain the photosensitive male sterile rice. The protein RMS1 is the following A1) or A2): A1), the amino acid sequence of which is as shown in SEQ ID No. 1 in the sequence listing; A2), a homologous protein having more than 98% identity with A1) and is derived from rice. The present invention obtains a rice photosensitive male sterile material by controlling the RMS1 gene of rice and its encoded protein, and achieves the fertility of rice under different light conditions.

### RELATED APPLICATIONS

The present application is a U.S. National Phase of International Application Number PCT/CN2021/089796, filed Apr. 26, 2021, and claims priority to Chinese Application Number 202010342657.1, filed Apr. 27, 2020, the contents of which are incorporated, in their entireties, by reference.

### INCORPORATION BY REFERENCE

The sequence listing provided in the file entitled PUS1221925-Revised_SQL.txt, which is an ASCII text file that was created on Dec. 21, 2022, and which comprises 30,263 bytes, is hereby incorporated by reference in its entirety.

### FIELD OF THE INVENTION

The present invention relates to the field of biotechnology breeding, in particular to a method for preparing a rice photosensitive male sterile material and related genes thereof.

### BACKGROUND OF THE INVENTION

Hybrid breeding technology has played a pivotal role in improving rice production in our country and the world and is an important guarantee for our country's food security and sustainable agricultural development. The two-line sterile line is freely matched and the seed production procedure is simplified, but its fertility is greatly affected by the external environment, and there are risks in seed production. The selection and breeding of photo-thermo-sensitive male sterile lines is the core of the development and application of the two-line hybrid rice breeding technology system. At present, the thermosensitive sterile line is widely used in production, which is mainly controlled by temperature. In a relatively changeable temperature environment, the length of light is more constant in a specific area, and the difference between years is smaller, therefore, screening photosensitive male sterile lines with stable fertility conversion has great application prospects in production.

In recent years, with the development of functional genomics research, important progress has been made in the research on the genetic system of male sterility and fertility restoration in rice.

YiD1S is a rice antiphotosensitive line, in which pollen fertility is mainly regulated by day length, but also affected by temperature. Genetic analysis revealed that male sterility of YiD1S was controlled by two recessive major genes (rpms1 and rpms2). rpms1 was mapped between SSR markers RM22980 (0.9 cM) and RM23017 (1.8 cM) on chromosome 8 and rpms2 was mapped between SSR markers RM23898 (0.9 cM) and YDS926 (0.9 cM) on chromosome 9. Subsequent studies found that the role of rpms1 was slightly greater than that of rpms2, and that the two genes interacted in the control of male sterility (Peng et al., 2008).

Pms1 is a photosensitive male nuclear sterile gene, and the Pms1 locus encodes a long non-coding RNA PMS1T. Pms1 is located between RFLP markers RG477 and RG511 on rice chromosome 7, with genetic distances of 3.5 cM and 15.0 cM, respectively (Zhang et al., 1994). It is finely localized in the 85 kb interval between R1807 and RG477 (Liu et al., 2001). PhasiRNAs (phased small interfering RNAs) produced by the Pms1 locus are associated with photoperiod-sensitive male sterility in rice. The Pms1 locus encodes a long non-coding RNA PMS1T, which is a target of miR2118. miR2118 acts on PMS1T to generate 21-nt phasiRNAs. PhasiRNAs are preferentially accumulated in photoperiod-sensitive male sterile lines under long-day conditions. A single-nucleotide polymorphism near the recognition site of miR2118 in PMS1T is critical for fertility changes and may lead to differential accumulation of phasiRNAs (Fan et al. 2016).

Ding et al. used Nongken 58S as the research material and cloned the gene pms3 that controls the photosensitive male sterility of this material, which controls a 1236 bp LDMAR (long non-coding RNA). Under long-day conditions, sufficient LDMAR transcription is necessary to maintain normal pollen development, but due to a single base mutation, the secondary structure of LDMAR is changed, and the methylation degree of its promoter region increased, which reduces the transcription amount of LDMAR under long-day conditions and causes the developing anther to enter the programming death in advance, resulting in photosensitive male sterility. Under short-day conditions, LDMAR is not necessary to maintain normal pollen development, thus shows fertile (Ding et al., 2012). Zhou et al. also cloned the pms3 gene using Pei'ai 64S (a derivative of Nongken 58S) as research material and named it p/tms12-1. The study found that p/tms12-1 encodes a specific non-coding RNA that can generate a small RNA of 21 nt, named osa-smR5864w. A point mutation in p/tms12-1 resulted in loss of function of osa-smR5864w, which turned into photosensitive and thermosensitive male sterility in indica and japonica rice, respectively (Zhou et al., 2012).

The mutation of the rice photosensitive male sterile gene CSA results in rice photosensitive male sterility, which specifically shows as short light sterility (defective sugar allocation in csa anthers and reduced sugar content in flower organs under short light conditions), long light fertility (sugar allocation defect in csa anthers is restored under long light conditions), and the F1 generation generated by crossing csa with restorer line JP69 has heterosis and can be used for rice hybrid rice seed production (Zhang et al. 2010, Zhang et al., 2013).

The rice thermosensitive male sterile gene TMS5 controls the thermosensitive male sterility of the rice thermosensitive sterile line An'nong S-1. This gene encodes a conserved RNase ZS1, which can process three ubiquitin ribosomal L40 fusion protein genes mRNA into multiple fragments in vivo and in vitro. Mutation of TMS5 results in loss of RNase ZS1 function and upregulation of UbL40 mRNA level at high temperature, and its excessive accumulation leads to reduced pollen yield and male sterility (Zhou et al., 2014). In addition, TMS5 is also a thermosensitive fertility gene that controls the thermosensitive male sterile materials indica S, Q523S, Q52S, N28S, G421S and Q527.

The rice thermosensitive male sterile gene TMS10 encodes a receptor-like kinase rich in leucine repeat sequence. tms10 showed high temperature male sterility and low temperature male fertility. Studies have shown that TMS10 plays an important role in the degradation of rice anther tapetum under high temperature conditions. TMS10 and its homologous gene TMS10L redundantly regulate rice anther development, and tms10 tms10l double mutants exhibited male sterility at both high and low temperature, suggesting that TMS10 gene specifically regulates rice anther development under high temperature conditions (Yu et al., 2017). However, in addition to the application of these genes in current production, there are still many genes that have not yet been discovered and control photo-thermo-sensitive male sterility. If these genes can be deeply explored and their functions can be studied, the knowledge and understanding of the mechanism of photo-thermo-sensitive male sterility will be more comprehensively expanded and deepened, and it will have important guiding significance and application value for breeding and creating new, excellent and stable two-line sterile lines.

### SUMMARY OF THE INVENTION

The technical problem to be solved by the present invention is how to prepare photosensitive male sterile rice.

In order to solve the above technical problems, the present invention first provides a method for preparing photosensitive male sterile rice.

The method for preparing photosensitive male sterile rice provided by the present invention includes: reducing the abundance of protein RMS1 in target rice, reducing the activity of protein RMS1 in target rice or reducing the content of protein RMS1 in target rice to obtain the photosensitive male sterile rice;

The protein RMS1 is the following A1) or A2):

A1), the amino acid sequence of which is as shown in SEQ ID NO. 1 in the sequence listing;

A2), a homologous protein having more than 98% or 99% identity with A1) and is derived from rice.

In the above proteins, the identity refers to the identity of the amino acid sequence. Amino acid sequence identity can be determined using homology search sites on the Internet, such as the BLAST page of the NCBI homepage website. For example, in advanced BLAST2.1, by using blastp as the program, the Expect value is set to 10, all Filters are set to OFF, BLOSUM62 is used as the Matrix, and the Gap existence cost, Per residue gap cost and Lambda ratio are set to 11, 1 and 0.85 (default value), respectively, and perform a search for the identity of a pair of amino acid sequences for calculation, and then the identity value (%) can be obtained.

The step of reducing the abundance of protein RMS1 in the target rice, reducing the activity of the protein RMS1 in the target rice or reducing the content of the protein RMS1 in the target rice is implemented by inhibiting the expression of the encoding gene of the protein RMS1 in the target rice or knocking out the encoding gene of the protein RMS1 in the target rice. The knockout includes knocking out an entire gene, and also includes knocking out a partial segment of the gene.

The step of “reducing the abundance of the protein RMS1 in the target rice, reducing the activity of the protein RMS1 in the target rice, or reducing the content of the protein RMS1 in the target rice” can also be implemented by silencing the encoding gene of the protein RMS1.

The step of “reducing the abundance of the protein RMS1 in the target rice, reducing the activity of the protein RMS1 in the target rice, or reducing the content of the protein RMS1 in the target rice” can be specifically implemented by gene editing of the gene encoding of the protein RMS1.

The encoding gene of the protein RMS1 is any one of the following b1)-b4):

b1), the nucleotide sequence of which is the DNA molecule shown in SEQ ID NO. 2 in the sequence listing;

b2), the its nucleotide sequence of which is the DNA molecule shown in SEQ ID NO. 3 in the sequence listing;

b3), a DNA molecule having 75% or more identity with the nucleotide sequence defined by b1) or b2) and encoding the protein RMS1;

b4), a DNA molecule hybridizing under strict conditions to the nucleotide sequence defined in b1) or b2) and encoding the protein RMS1.

In the above genes, “identity” refers to the sequence similarity with the natural nucleic acid sequence. “Identity” includes a nucleotide sequence that is 75% or more, or 85% or more, or 90% or more, or 95% or more identical to the nucleotide sequence of the protein composed of the amino acid sequence encoding sequence 2 of the present invention. Identity can be assessed with the naked eye or with computer software. Using computer software, the identity between two or more sequences can be expressed in percentage (%), which can be used to assess the identity between related sequences.

The strict conditions are hybridization and membrane washing twice in a solution of 2×SSC, 0.1% SDS at 68° C., 5 min each time, and hybridization and membrane washing twice in a solution of 0.5×SSC, 0.1% SDS at 68° C., 15 min each time; or hybridization and membrane washing in a solution of 0.1×SSPE (or 0.1×SSC) and 0.1% SDS at 65° C.

The above 75% or more identity may be 80%, 85%, 90% or more than 95% identity.

In the above method, any method in the prior art can be used to inhibit the expression of the RMS1 gene or to knockout the RMS1 gene, so as to generate deletion mutation, insertion mutation or base change mutation in the gene, thereby inhibiting RMS1 gene expression or knockout of the RMS1 gene.

In the above method, to inhibit the expression of the encoding gene of the protein RMS1 in the target rice or knockout the encoding gene of the protein RMS1 in the target rice, chemical mutagenesis, physical mutagenesis, RNAi, gene site-directed editing, homologous recombination and other methods can be adopted.

Whichever method is taken, the RMS1 gene can be used as target, as well as individual elements that regulate RMS1 gene expression, as long as RMS1 gene expression can be inhibited or RMS1 gene knocked out. For example, exon 1, exon 2, exon 3 and/or exon 4 of the RMS1 gene can be used as targets.

In the above genome site-directed editing, zinc finger nuclease (Zinc finger nuclease, ZFN) technology, transcription activator-like effector nuclease (Transcription activator-like effector nuclease, TALEN) technology or clustered regularly interspaced short palindromic repeats and related systems (Clustered regularly interspaced short palindromic repeats/CRISPR associated, CRISPR/Cas9 system) technology and other technologies that can achieve genome site-directed editing can be used.

In a specific embodiment of the present invention, the CRISPR/Cas9 system is used to achieve the knockout of the encoding gene of the protein RMS1 in rice, wherein the involved target sequence is CCAAGGCCGGTAAGCGCCGC (SEQ ID NO. 15), and the encoding gene of the sgRNA (guide RNA) used is as shown in SEQ ID NO. 4 in the sequence listing.

More specifically, a recombinant vector pYLCRISPR/Cas9-MT-RMS1 capable of expressing guide RNA and Cas9 is used in the present invention. The recombinant vector pYLCRISPR/Cas9-MT-RMS1 is a recombinant vector obtained by replacing the fragment between the two BsaI restriction sites of the vector pYLCRISPR/Cas9-MTmono with a DNA fragment containing a specific sgRNA encoding gene and U3 promoter and maintaining the other nucleotides of pYLCRISPR/Cas9-MTmono unchanged. It was obtained specifically by replacing the fragment between two BsaI restriction sites of pYLCRISPR/Cas9-MTmono with the DNA molecule shown in SEQ ID NO. 5 in the sequence listing. The above method is applicable to any rice variety, such as: Oryza sativa subsp. japonica or Oryza sativa subsp. indica, as long as it contains the above target sequence. An example exemplified by the present invention is the rice variety Wuyunjing 7 (Oryza sativa subsp. japonica).

The present invention also protects specific sgRNAs. The target sequence of the specific sgRNA is as follows: CCAAGGCCGGTAAGCGCCGC (SEQ ID NO 15).

The present invention also protects a specific recombinant plasmid. The specific recombinant plasmid is pYLCRISPR/Cas9-MT-RMS1.

pYLCRISPR/Cas9-MT-RMS1 contains the gene encoding Cas9 protein and the gene encoding sgRNA.

The present invention also protects use of the above specific sgRNA or specific recombinant plasmid in rice breeding; the purpose of the rice breeding is to cultivate photosensitive male sterile rice.

The present invention also protects a method for preparing a transgenic plant, comprising the following steps: introducing the encoding gene of sgRNA and the encoding gene of Cas9 protein into recipient rice to obtain photosensitive male sterile rice. The encoding gene of the specific sgRNA and the encoding gene of the Cas9 protein are specifically introduced into the recipient rice through the recombinant plasmid.

In order to solve the above technical problems, the present invention also provides a protein RMS1.

The protein RMS1 is the following A11) or A12):

A11), the amino acid sequence of which is as shown in SEQ ID NO. 1 in the sequence listing;

A12), a homologous protein having more than 98% or 99% identity with A11) and is derived from rice;

wherein, the protein shown in SEQ ID NO. 1 consists of 345 amino acid residues.

In order to solve the above technical problem, the present invention also provides a gene encoding protein RMS1.

The gene encoding protein RMS1 provided by the present invention is any one of the following b11)-b14):

b11), the nucleotide sequence of which is the DNA molecule shown in SEQ ID NO. 2 in the sequence listing;

b12), the nucleotide sequence of which is the DNA molecule shown in SEQ ID NO. 3 in the sequence listing;

b13), a DNA molecule having 75% or more identity with the nucleotide sequence defined in b11) or b12) and encoding protein RMS1;

b14), a DNA molecule that hybridizes under strict conditions to the nucleotide sequence defined in b11) or b22) and encodes protein RMS1.

wherein, SEQ ID NO. 2 in the sequence listing consists of 1038 nucleotides, and encodes the protein shown in SEQ ID NO. 1 in the sequence listing.

In order to solve the above technical problems, the present invention also provides a protein RMS1-4.

The protein RMS1-4 is the following A21) or A22):

A21), the amino acid sequence of which is as shown in SEQ ID NO. 6 in the sequence listing;

A22), a homologous protein having more than 98% or 99% identity with A21) and is derived from rice.

Wherein, the protein shown in SEQ ID NO. 6 consists of 359 amino acid residues.

In order to solve the above technical problems, the present invention also provides a gene encoding protein RMS1-4.

The gene encoding protein RMS1-4 provided by the present invention is any one of the following b21)-b24):

b21), the nucleotide sequence of which is the DNA molecule shown in SEQ ID NO. 7 in the sequence listing;

b22), the nucleotide sequence of which is the DNA molecule shown in SEQ ID NO. 8 in the sequence listing;

b23), a DNA molecule having 75% or more identity with the nucleotide sequence defined in b21) or b22) and encoding the protein RMS1-4;

b24), a DNA molecule that hybridizes to the nucleotide sequence defined in b21) or b22) under strict conditions and encodes the protein RMS1-4.

Wherein, SEQ ID NO. 7 in the sequence listing consists of 1080 nucleotides, encoding the protein shown in SEQ ID NO. 6 in the sequence listing.

In order to solve the above technical problems, the present invention also provides a protein RMS1-5.

The protein RMS1-5 is the following A31) or A32):

A31), the amino acid sequence of which is as shown in SEQ ID NO. 9 in the sequence listing;

A32), a homologous protein having more than 98% or 99% identity with A31) and is derived from rice.

Wherein, the protein shown in SEQ ID NO. 9 consists of 111 amino acid residues.

In order to solve the above technical problems, the present invention also provides a gene encoding protein RMS1-5.

The gene encoding protein RMS1-5 provided by the present invention is any one of the following b31)-b34):

b31), the nucleotide sequence of which is the DNA molecule shown in SEQ ID NO. 10 in the sequence listing;

b32), the nucleotide sequence of which is the DNA molecule shown in SEQ ID NO. 11 in the sequence listing;

b33), a DNA molecule having 75% or more identity with the nucleotide sequence defined in b31) or b32) and encoding protein RMS1-5;

b34), a DNA molecule that hybridizes to the nucleotide sequence defined in b31) or b32) under strict conditions and encodes protein RMS1-5.

Wherein, SEQ ID NO. 10 in the sequence listing consists of 336 nucleotides, encoding the protein shown in SEQ ID NO. 9 in the sequence listing.

In order to solve the above technical problems, the present invention provides a protein RMS1-11.

The protein RMS1-11 is the following A41) or A42):

A41), the amino acid sequence of which is as shown in SEQ ID NO. 12 in the sequence listing;

A42), a homologous protein having 98% or more identity with A41) and is derived from rice.

Wherein, the protein shown in SEQ ID NO. 12 consists of 360 amino acid residues.

In order to solve the above technical problem, the present invention also provides a gene encoding protein RMS1-11.

The gene encoding protein RMS1-11 provided by the present invention is any one of the following b1)-b4):

b41), the nucleotide sequence of which is the DNA molecule shown in SEQ ID NO. 13 in the sequence listing;

b42), the nucleotide sequence of which is the DNA molecule shown in SEQ ID NO. 14 in the sequence listing;

b43), a DNA molecule having 75% or more identity with the nucleotide sequence defined in b41) or b42) and encoding the protein RMS1-11;

b44), a DNA molecule that hybridizes to the nucleotide sequence defined in b41) or b42) under strict conditions and encodes the proteins RMS1-11.

Wherein, SEQ ID NO. 13 in the sequence listing consists of 1083 nucleotides, encoding the protein shown in SEQ ID NO. 12 in the sequence listing.

In order to solve the above technical problems, the present invention also provides the use of RMS1 protein or the encoding gene of the RMS1 protein in regulating the rice photoperiod-sensitive fertility.

In order to solve the above technical problem, the present invention also provides the use of RMS1 protein or the encoding gene of the RMS1 protein in regulating the photosensitive male sterile rice.

In order to solve the above technical problem, the present invention also provides the cultivation of photosensitive male sterile rice by using the RMS1 protein or the encoding gene of the RMS1 protein as a target.

In the present invention, the male sterility is embodied as decreased pollen fertility or pollen abortion.

In the present invention, the photosensitive male sterility is the photo-thermo-sensitive male sterility in which photo-sensitivity plays a leading role; the photo-thermo-sensitive male sterility is photo-thermo-sensitive nuclear male sterility.

In the present invention, the photosensitive male sterile rice is the rice whose pollen fertility changes with the change of the light time.

### BRIEF DESCRIPTION OF THE FIGURES

FIG. 1 is a map of the intermediate vector pYLgRNA-U3.

FIG. 2 is the electrophoresis detection graph of the expression cassette amplification of intermediate vector pYLgRNA-U3-RMS1.

FIG. 3 is a map of the genome editing vector pYLCRISPR/Cas9-MTmono vector.

FIG. 4 is an electrophoresis diagram of the PCR detection results of the monoclonal colonies of Escherichia coli transformed with the recombinant vector pYLCRISPR/Cas9-MT-RMS1.

FIG. 5 is a sequence alignment diagram of the recombinant vector pYLCRISPR/Cas9-MT-RMS1.

FIG. 6 shows the type of RMS1 gene mutation and the type of amino acid encoded after mutation; wherein, 9522^{38740-target} is wild-type rice 9522; the black part of the encoded protein is the RMS1 core domain, and the gray part is the newly encoded protein region after RMS1 mutation.

FIG. 7 is the population phenotypic analysis of the F₂ hybrid between the wild-type rice variety 9522 and the RMS1 homozygous mutant 9522³⁸⁷⁴⁰⁻⁵.

FIG. 8 is the phenotype comparison between the RMS1 homozygous mutant 9522³⁸⁷⁴⁰⁻⁵ and the wild-type rice variety 9522 under different light durations; A is the long light treatment, and B is the short light treatment.

FIGS. 9A-9D show the comparison of the 12-KI staining effect of pollen grains between RMS1 homozygous mutant 9522³⁸⁷⁴⁰⁻⁵ and wild-type rice variety 9522 under short-day light and different temperature treatments; wherein, FIG. 9A is the staining microscopic examination of pollen grains of wild-type rice variety 9522 after short-day high temperature treatment, FIG. 9B is the staining microscopic examination of pollen grains of homozygous mutant 9522³⁸⁷⁴⁰⁻⁵ after short-day high temperature treatment, FIG. 9C is the staining microscopic examination of pollen grains of wild-type rice variety 9522 after short-day low temperature treatment, and FIG. 9D is the staining microscopic examination of pollen grains of homozygous mutant 9522³⁸⁷⁴⁰⁻⁵ after short-day low temperature treatment.

FIG. 10 shows the pollen fertility changes of RMS1 homozygous mutant 9522³⁸⁷⁴⁰⁻⁵ and wild-type rice variety 9522 grown in different natural ecological regions.

### DETAILED DESCRIPTION

The experimental methods used in the following examples are conventional methods unless otherwise specified.

The materials, reagents, etc. used in the following examples are commercially available unless otherwise specified.

The expression vector pYLgRNA-U3 is described in the literature “Shi Jiangwei, Li Yixing, Song Shufeng, Qiu Peony, Deng Yao, Li Li, Targeted Editing of Rice Panicle Development Gene Osal Mediated by CRISPR/Cas9 System. HYBRID RICE, 2017, 32(3): 74-78,” which can be obtained by the public from the Hunan Hybrid Rice Research Center, and this biological material is only used for repeating the relevant experiments of the present invention and cannot be used for other purposes.

The expression vector pYLCRISPR/Cas9-MTmono is described in the literature “Shi Jiangwei, Li Yixing, Song Shufeng, Qiu Peony, Deng Yao, Li Li, Targeted Editing of Rice Panicle Development Gene Osal Mediated by CRISPR/Cas9 System. HYBRID RICE, 2017, 32(3): 74-78,” which can be obtained by the public from the Hunan Hybrid Rice Research Center, and the biological material is only used for repeating the relevant experiments of the present invention and cannot be used for other purposes.

The rice variety Wuyunjing 7 (original code 9522) has been disclosed in the document “CRISPR/Cas9-directed editing of the Osal gene of rice panicle development. Hybrid Rice (HYBRID RICE), 2017, 32(3): 74-78,” which can be obtained by the public from the Hunan Hybrid Rice Research Center, and the biological material is only used for repeating the relevant experiments of the present invention and cannot be used for other purposes.

### Example 1 Selection of Target Site of Rice RMS1 Gene and Construction of Knockout Vector

The inventors of the present invention discovered a gene related to photosensitive male sterility, the RMS1 (Reverse Male Sterility) gene from rice Wuyunjing 7. The encoding sequence of the RMS1 gene was shown in SEQ ID NO. 2 in the sequence listing, which encoded a protein RMS1 consisting of 345 amino acid residues, and its amino acid sequence was shown in SEQ ID NO. 1 in the sequence listing. The full-length gDNA of the RMS1 gene was 2623 bp, containing 3 exons and 4 introns, and its nucleotide sequence was shown in SEQ ID NO. 3 in the sequence listing.

In this example, the rice RMS1 gene was knocked out by CRISPR/Cas9 gene editing technology to obtain mutants 9522³⁸⁷⁴⁰⁻⁴, 9522³⁸⁷⁴⁰⁻⁵ and 9522³⁸⁷⁴⁰⁻¹¹ with photosensitive male sterile phenotype, and mutant 9522³⁸⁷⁴⁰⁻⁴, 9522³⁸⁷⁴⁰⁻⁵ and 9522³⁸⁷⁴⁰⁻¹¹ were all RMS1 knockout rice. The specific operation method was as follows:

1. Design of a Target Sequence

The target sequence used was 5′-CCAAGGCCGGTAAGCGCCGC-3′ (SEQ ID NO. 15), which was located at the junction of the first exon and the second intron sequence, that is, positions 384 to 403 of the sequence 3.

2. Construction of Intermediate Vector pYLgRNA-U3-RMS1

(1) Design and Synthesis of RMS1 Target Site Linker Primer

After the target site sequence was determined, GGCA was added before the 5′ of the sense strand of the target sequence, and AAAC was added before the 5′ of the antisense strand to obtain the target site linker primer. The target site linker primer sequences were as follows:

RMS1-Cas9-F:
(SEQ ID NO. 16)
5′-GGCACCAAGGCCGGTAAGCGCCGC ′;
RMS1-Cas9-R:
(SEQ ID NO. 17)
5′-AAACGCGGCGCTTACCGGCCTTGG-3′.

(2) Preparation of RMS1 Target Site Linkers

The RMS1 target site linker primers RMS1-Cas9-F and RMS1-Cas9-R were diluted with ddH₂O to stock solutions with a concentration of 10 μM. 10 μL to 80 μL deionized water was taken for each solution to a final volume of 100 μL. The solution was fully mixed and then heat-shocked at 90° C. for 30s, and then moved to room temperature to complete the annealing and the RMS1 target site linker was obtained, which was labeled as RMS1-Cas9.

(3) Construction of RMS1 Intermediate Vector

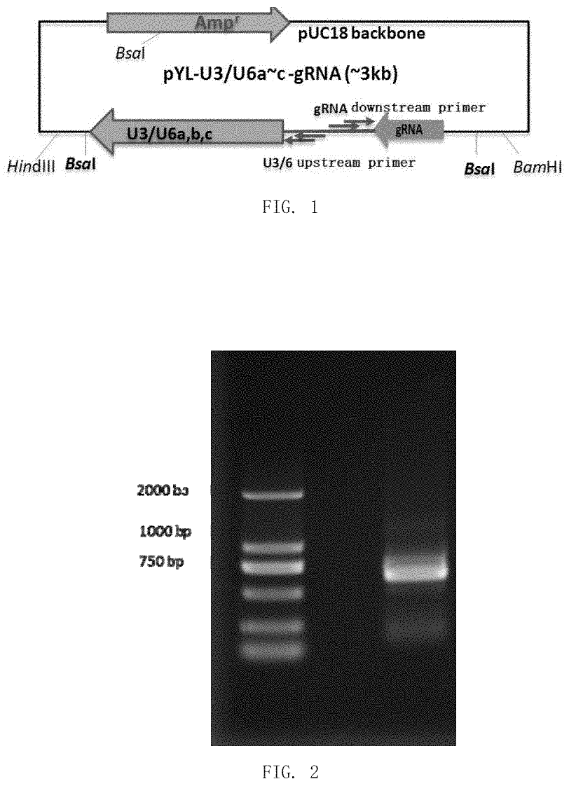

1 μL pYLgRNA-U3 vector plasmid (as shown in FIG. 1), 1 μL 10×T4 DNA Ligase Buffer, 1 μL target site linker RMS1-Cas9, 1 μL BsaI restriction endonuclease and 0.5 μL 10×T4 DNA Ligase were mixed evenly. The PCR instrument was used for the reaction, and the reaction conditions were 37° C. for 5 min, 20° C. for 5 min, 5 cycles, thereby obtaining an intermediate vector containing the target sequence of the rice RMS1 gene, which was named pYLgRNA-U3-RMS1.

3. Construction and Transformation of RMS1 Site-Directed Editing Final Vector

(1) Amplification of RMS1 Intermediate Vector Expression Cassette

The intermediate vector pYLgRNA-U3-RMS1 was used as the template, Uctcg-B1: TTCAGAGGTCTCTCTCGCACTGGAATCGGCAGCAAAGG (SEQ ID NO. 18) and gRcggt-BL: AGCGTGGGTCTCGACCGGGTCCATCCACTCCAAGCTC (SEQ ID NO. 19) were used as primers for PCR amplification, and the amplified products were obtained. The amplified products were detected by gel electrophoresis, and it was determined as a DNA molecule with a size of about 550 bp (as shown in FIG. 2). The amplification result was consistent with the expectation. The amplified products were recovered and purified and named as the RMS1 intermediate vector expression cassette. The expression cassette contained a sgRNA encoding gene and a U3 promoter, wherein the sgRNA target sequence was 5′-CCAAGGCCGGTAAGCGCCGC-3′ (SEQ ID NO. 15), and the sgRNA encoding gene was shown in SEQ ID NO. 4 in the sequence listing.

(2) Construction and Transformation of RMS1 Site-Directed Editing Final Vector

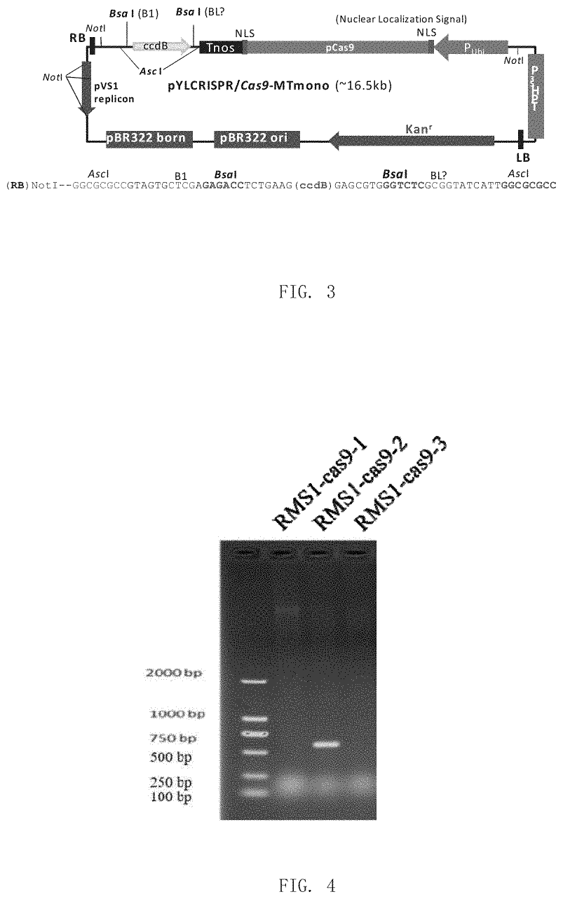

Using BsaI restriction endonuclease and T4 DNA Ligase, the gene editing vector pYLCRISPR/Cas9-MTmono (as shown in FIG. 3) and the RMS1 intermediate vector expression cassette were digested and ligated to obtain the RMS1 gene site-directed editing final vector pYLCRISPR/Cas9-MT-RMS1. E. coli was transformed, spread on a plate containing kanamycin, and cultured at 37° C. overnight.

(3) Detection of Recombinant Vector pYLCRISPR/Cas9-MT-RMS1

Three monoclonal colonies cultivated overnight in step (2) were randomly selected and named as RMS1-cas9-1, RMS1-cas9-2, and RMS1-cas9-3, respectively. Three monoclonal colonies were detected by PCR using the pYLCRISPR/Cas9-MTmono vector: SP1: CCCGACATAGATGCAATAACTTC (SEQ ID NO. 20) and SP2: GCGCGGTGTCATCTATGTTACT (SEQ ID NO. 21). The PCR amplification products were subjected to gel electrophoresis, and the electrophoresis results (as shown in FIG. 4) showed that the RMS1-cas9-2 monoclonal colony could amplify a band with a size of about 550 bp, which was consistent with the expectation.

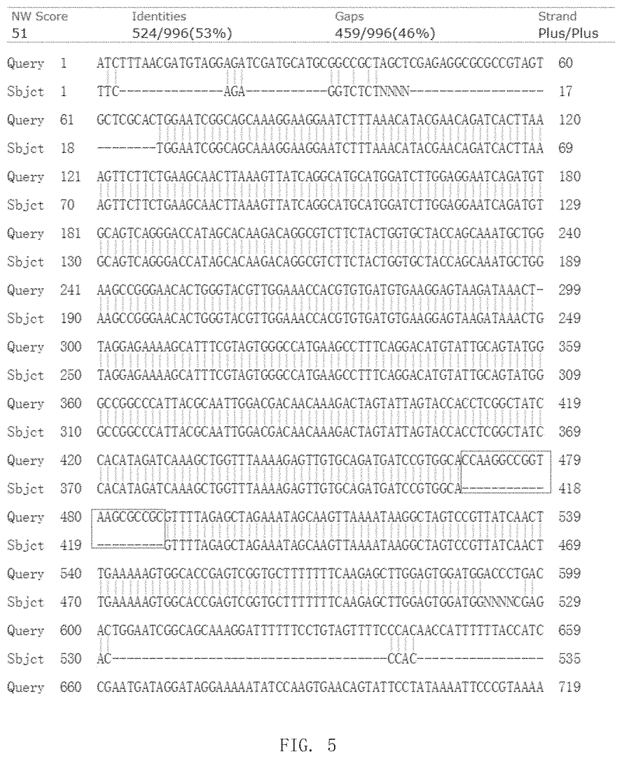

Plasmid DNA of RMS1-cas9-2 monoclonal was extracted and sequenced. The sequencing results (as shown in FIG. 5) showed that the DNA fragment shown in SEQ ID NO. 5 in the sequence listing successfully replaced the DNA fragment between the two BsaI restriction sites on the gene editing vector pYLCRISPR/Cas9-MTmono. This indicated that the expression cassette containing the U3 promoter and the sgRNA encoding gene was successfully constructed into the gene editing vector pYLCRISPR/Cas9-MTmono, that is, the genome site-directed editing vector of RMS1 was successfully constructed, and the recombinant vector pYLCRISPR/Cas9-MT-RMS1 was obtained.

### Example 2 Acquisition and Phenotypic Analysis of RMS1 Mutant Rice Material

1. Acquisition of RMS1 Mutant Rice Material

Using the method of Agrobacterium-mediated transformation of rice callus, the RMS1 gene site-directed editing vector pYLCRISPR/Cas9-MT-RMS1 was used to transform the callus of rice variety Wuyunjing 7 (original code 9522, hereinafter referred to as 9522), and positive mutants were screened and identified.

2. The Detection of Fixed-Point Editing

Three homozygous mutants with knockout of RMS1 gene were obtained by PCR detection, which were named as homozygous mutant 9522³⁸⁷⁴⁰⁻⁴, homozygous mutant 9522³⁸⁷⁴⁰⁻⁵, and homozygous mutant 9522³⁸⁷⁴⁰⁻¹¹ The sequencing results showed (as shown in FIG. 6):

The RMS1 gene (wild-type) was mutated in the homozygous mutant 9522³⁸⁷⁴⁰⁻⁴, and 2 bases were deleted from the 128th-129th position of the CDS of the RMS1 gene. This mutation caused the ORF of the RMS1 gene to shift after the 126th position, and a new stop codon was formed in the RMS1 3′UTR sequence. The gene after the frame shift mutation was named RMS1-4 gene, the nucleotide sequence of RMS1-4 gene was shown in SEQ ID NO. 8 in the sequence listing, and the encoding sequence of RMS1-4 gene was shown in SEQ ID NO. 7 in the sequence listing, which encoded a protein RMS1-4 composed of 359 amino acid residues, and its amino acid sequence was shown in SEQ ID NO. 6 in the sequence listing.

The RMS1 gene (wild-type) was also mutated in the homozygous mutant 9522³⁸⁷⁴⁰⁻⁵, and 1 base was deleted from the 127th position of the CDS of the RMS1 gene. This mutation caused the ORF of the RMS1 gene to shift after the 126th position, and a new stop codon was formed in advance at 334-336 bp of the CDS of the RMS1 gene to terminate translation. The gene after the frame shift mutation was named RMS1-5 gene. The nucleotide sequence of RMS1-5 gene was shown in SEQ ID NO. 11 in the sequence listing, the encoding sequence of RMS1-5 gene was shown in SEQ ID NO. 10 in the sequence listing, which encoded a protein RMS1-5 consisting of 111 amino acid residues, and its amino acid sequence was shown in SEQ ID NO. 9 in the sequence listing.

The RMS1 gene (wild-type) was mutated in the homozygous mutant 9522³⁸⁷⁴⁰⁻¹¹, and 1 base was inserted into the 127th position of the CDS of the RMS1 gene. This mutation caused the ORF of the RMS1 gene to shift after the 126th position, and a new stop codon was formed in the RMS1 3′ UTR sequence. The gene after the frame shift mutation was named RMS1-11 gene. The nucleotide sequence of RMS1-11 gene was shown in SEQ ID NO. 14 in the sequence listing, the encoding sequence of the RMS1-11 gene was shown in SEQ ID NO. 13 in the sequence listing, which encoded a protein RMS1-11 composed of 360 amino acid residues, and its amino acid sequence was shown in SEQ ID NO. 12 in the sequence listing.

The two core domains SANT of the wild-type RMS1 protein were located at positions 14-61 and 67-112 of the amino acid sequence, respectively. It can be seen that the mutations at the above sites led to the deletion of the core domain SANT, which in turn affected the function of the RMS1 gene.

3. Construction and Phenotypic Analysis of RMS1 Mutant F₂ Co-Segregation Population

The homozygous mutants 9522³⁸⁷⁴⁰⁻⁴, 9522³⁸⁷⁴⁰⁻⁵, 9522³⁸⁷⁴⁰⁻¹¹ had the same mutant phenotype and agronomic traits. Therefore, in the subsequent examples of the present invention, the homozygous mutant 9522³⁸⁷⁴⁰⁻⁵, was used as an example for detailed phenotypic analysis.

(1) F₂ Group Construction

The wild-type 9522 was used as the female parent, the T₁ generation single plant obtained from the homozygous mutant 9522³⁸⁷⁴⁰⁻⁵ was used as the male parent for hybridization (planting time: 201806-201810), and the F₁ hybrid seeds were harvested. The F₁ generation population was planted in Lingshui, Hainan (planting time: 201812-201904). F₁ generation population was self-crossed to obtain F₂ generation population, and the F₂ generation population was planted in Changsha, Hunan (planting time: 201906-201910).

(2) Phenotypic Analysis

There were a total of 42 individual plants in the F₂ generation isolated population in step 1. After genotype identification and analysis of all the individual plants in the F₂ isolated population, a total of 3 genotypes were isolated, which were the wild-type genotype (the corresponding positions of the two chromatids were both from the RMS1 gene of wild-type rice) and the RMS1 mutant material homozygous mutant 9522³⁸⁷⁴⁰⁻⁵ homozygous genotype (the corresponding positions of the two chromosomes were the RMS1-5 gene from the homozygous mutant 9522³⁸⁷⁴⁰⁻⁵, hereinafter referred to as the 9522³⁸⁷⁴⁰⁻⁵ genotype), and the heterozygous genotype obtained by crossing the wild-type and RMS1 mutant material homozygous mutant 9522³⁸⁷⁴⁰⁻⁵ genotype (that is, the corresponding position of one chromosome was the RMS1 gene from wild-type rice, and the corresponding position of the other chromosome was the RMS1-5 gene from the homozygous mutant 9522³⁸⁷⁴⁰⁻⁵, hereinafter referred to as the heterozygous genotype). Wherein, there were 7 strains in the wild-type genotype group, 26 strains in the heterozygous genotype group, and 9 strains in the 9522³⁸⁷⁴⁰⁻⁵ genotype group. The separation ratio of wild-type genotype:heterozygous genotype:9522³⁸⁷⁴⁰⁻⁵ genotype was 7:26:9, generally in line with the separation ratio of 1:2:1.

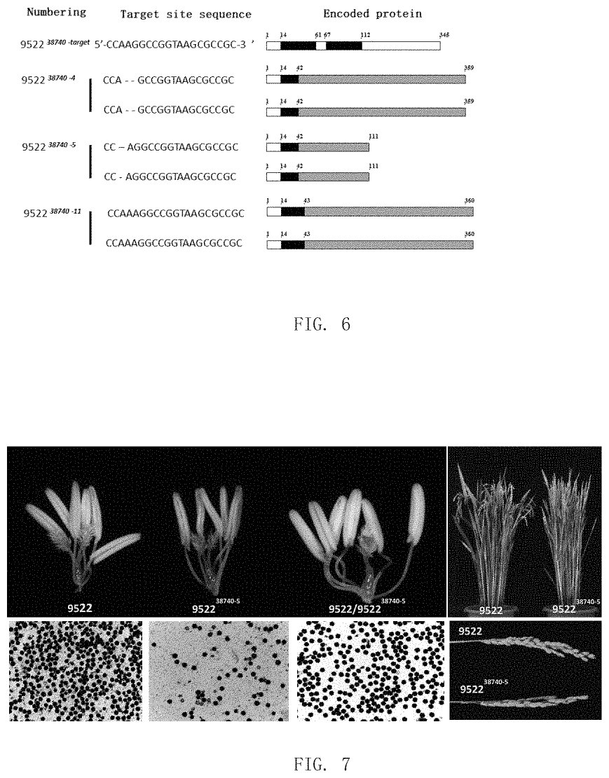

Planting observation found that the leaf morphology of each individual plant in the F₂ generation segregated population was consistent. Through microscopic observation, it was found that the anther morphology of different genotypes was different. In the segregating population of F₂ generation, the wild-type population had bright yellow anthers, full anther shape, normal number of pollen grains and anther microscopically fertile. The heterozygous genotype population was consistent with the wild-type population. However, in the 9522³⁸⁷⁴⁰⁻⁵ genotype population, the anthers were abnormally whitened, the morphology of anthers were shriveled, the number of pollen grains suddenly decreased, and the microscopic examination of anthers showed that there were a lot of sterile pollen grains (as shown in FIG. 7). Corresponding to the segregation ratio of F₂ population, it indicated that the mutation of RMS1 gene led to abnormal anther development and thus affected the pollen fertility of recipient plants.

### Example 3 Analysis of Photosensitive Characteristics of RMS1 Mutant Rice Materials

1. Analysis of Photosensitive Characteristics of RA/S1 Mutant Rice Materials

The homozygous mutant 9522³⁸⁷⁴⁰⁻⁴T₂ generation, the homozygous mutant 9522³⁸⁷⁴⁰⁻⁵T₂ generation and the wild-type rice 9522 of RMS1 mutant material were planted under natural conditions in Lingshui, Hainan (18° 51′23″N, 110° 5′6″E). The results showed that the homozygous mutant 9522³⁸⁷⁴⁰⁻⁴T₂ generation and the homozygous mutant 9522³⁸⁷⁴⁰⁻⁵T₂ generation plants had low seed setting rate, which were 4.56% and 3.13%, respectively, while the seed setting rate of wild-type rice 9522 was 95.6% (201812-201904); the homozygous mutant 9522³⁸⁷⁴⁰⁻⁴T₃ generation, the homozygous mutant 9522³⁸⁷⁴⁰⁻⁵T₃ generation and the wild-type rice 9522 were planted under natural conditions in Changsha, Hunan (28° 13′07″N, 113° 15′10″E). The results showed that the homozygous mutant 9522³⁸⁷⁴⁰⁻⁴T₃ generation and the homozygous mutant 9522³⁸⁷⁴⁰⁻⁵T₃ generation mutant were 35.29% and 16.02%, respectively, while the seed setting rate of wild-type rice 9522 was 96.75% (201906-201910). The same RMS1 mutant rice line had obvious differences in the seed setting rate in different regions. It was believed that the length of light in different regions would affect the fertility of pollen, which will lead to changes in the seed setting rate.

In order to further explore the effect of light on the pollen fertility of RMS1 mutant plants, 7 homozygous mutant 9522³⁸⁷⁴⁰⁻⁵ T₃ generation plants were subjected to exploratory short light treatment (light duration 10.5 hours, light intensity 30000 Lx; the temperature in the light period was 30° C., and the temperature in the dark period was 24° C.) and long light treatment (light duration 13.5 hours, light intensity 30,000 Lx; the temperature in the light duration was 30° C., and the temperature in the dark period was 24° C.) experiments. A11 experiments were carried out in a greenhouse with controlled temperature and light. Wild-type rice 9522 was used as the control.

Mature anthers of wild-type rice 9522 and mutant 9522³⁸⁷⁴⁰⁻⁵ T₃ generation plants were collected for microscopy and iodine staining. During iodine staining, the anthers of 3 florets of a single rice plant were taken and placed on a glass slide, the anthers were smashed with tweezers to release the pollen grains, and 1-2 drops of I₂-KI solution were added and covered with a cover glass for observation under a microscope. Those that were dyed blue were the pollen grains with strong vitality, and those that were yellow-brown were stunted pollen grains. Three fields of view were taken from each film, and the pollen staining rate was counted to represent the pollen fertility.

The results showed that, under short light conditions, the anthers of wild-type rice 9522 were bright yellow, plump in shape and normal in number of pollen grains. Pollen iodine staining showed a staining rate of 95.64%, indicating that the pollen fertility of wild-type rice 9522 was normal. Under the same conditions, the anthers of the homozygous mutant 9522³⁸⁷⁴⁰⁻⁵ T₃ generation plants were white and shriveled, and the number of pollen grains decreased significantly. The pollen iodine staining rate was only 28.17%, indicating that the pollen fertility of the mutant 9522³⁸⁷⁴⁰⁻⁵ T₃ generation plants decreased significantly (as shown in Table 1 and FIG. 8B).

Under long light conditions, the anthers of wild-type rice 9522 were bright yellow, plump in shape, and normal in number of pollen grains. The pollen was stained with iodine, and the staining rate was 96.18%, indicating that the pollen fertility of wild-type rice 9522 was normal. Under the same conditions, the anthers of the homozygous mutant 9522³⁸⁷⁴⁰⁻⁵ T₃ generation plants were bright yellow, plump in shape, and the number of pollen grains was normal. The pollen was stained with iodine, and the staining rate was 86.40% (as shown in Table 1 and FIG. 8A).

Compared with short light conditions, the number and staining rate of pollen grains of the homozygous mutant 9522³⁸⁷⁴⁰⁻⁵ T₃ generation plants were significantly restored under long light conditions, indicating that the pollen fertility of the homozygous mutant 9522³⁸⁷⁴⁰⁻⁵ T₃ generation plants was restored.

TABLE 1
Statistics of I₂-KI staining results of pollen grains of RMS1
homozygous mutant 9522³⁸⁷⁴⁰⁻⁵T₃ and wild-type rice variety
9522 under different light conditions

| | | Total | Number of | |
|---|---|---|---|---|
| | | number of | non-stained | |
| | | pollen | pollen | Staining |
| Treatment | Treatment | grains | grains | rate |
| conditions | materials | (grain) | (grain) | (%) |
| Short light | 9522 | 1838 | 80 | 95.64 |
| conditions | Homozygous | 323 | 232 | 28.17** |
| | mutant | | | |
| | 9522³⁸⁷⁴⁰⁻⁵ | | | |
| Long light | 9522 | 1859 | 71 | 96.18 |
| conditions | Homozygous | 1920 | 261 | 86.40 |
| | mutant | | | |
| | 9522³⁸⁷⁴⁰⁻⁵ | | | |

Note:
**P < 0.01.

It can be seen that under different light conditions, the anther color, morphology and pollen fertility of the control material 9522 were consistent, indicating that the light duration did not affect the pollen fertility of the recipient material 9522. However, the pollen of the RMS1 homozygous mutant 9522³⁸⁷⁴⁰⁻⁵ T₃ generation plants showed significant differences in anther color, morphology and number of pollen grains under different light duration treatments. Under short light conditions, the anthers of the homozygous mutant 9522³⁸⁷⁴⁰⁻⁵T₃ generation plants were white in color and shriveled in shape. The results of iodine staining showed that the number of pollen grains was greatly reduced, and there were a large number of sterile pollen grains, which was significantly different from the control under the same conditions. Under long light conditions, the number of pollen grains and fertility of the homozygous mutant 9522³⁸⁷⁴⁰⁻⁵ T₃ generation plants were significantly restored, and the anther color, shape and number of pollen grains of the homozygous mutant 9522³⁸⁷⁴⁰⁻⁵ T₃ generation plants were consistent with the control material under the same conditions. The results indicated that the pollen fertility of RMS1 mutant materials was sensitive to the light duration, and the pollen fertility of RMS1 mutants decreased sharply under short light conditions, while the pollen fertility of RMS1 mutants could be restored under long light conditions.

2. The Effect of Temperature on the Pollen Fertility of RMS1 Mutant Rice

(1) Effects of Temperature on the Fertility of RMS1 Mutant Rice Under Greenhouse Conditions

In order to further explore the relationship between pollen fertility and temperature of RMS1 mutant materials, different temperatures were set on the basis of 12-hour short light conditions.

The 6 9522³⁸⁷⁴⁰⁻⁵ T₃ generation plants and 6 wild-type 9522 plants grown in the field were transferred respectively to the culture pot during the jointing period to ease the growth of the plants. In the early booting stage, the plants were moved into an incubator for short light, high-low temperature treatment. Wherein, the treatment conditions of short light and low temperature (hereinafter referred to as short-day low temperature) were 12 hours of light duration, light intensity of 30000 Lx, and temperature of 23° C.; the conditions of short light high temperature (hereinafter referred to as short-day high temperature) treatment were 12 hours of light duration, 30,000 Lx of light intensity, and 33° C. of temperature. After heading, the pollen microscopic examination was carried out on the plants under different temperature treatments. 3 florets were taken from each individual plant and mixed for microscopic examination. The microscopic examination method of pollen fertility was the same as above. Three fields of view were taken from each film, and the pollen staining rate was counted to express the pollen fertility.

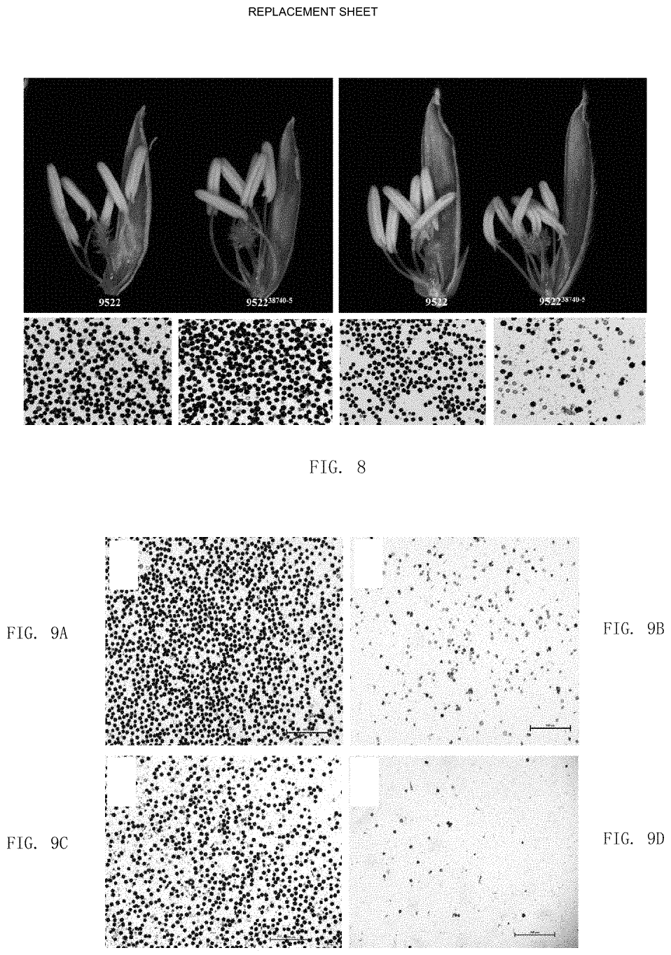

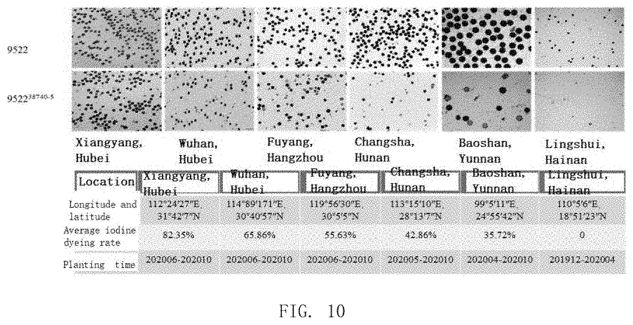

The results showed that under short-day high temperature conditions, the pollen iodine staining rate of wild-type rice 9522 was 94.41%, and the pollen iodine staining rate of 9522³⁸⁷⁴⁰⁻⁵ T₃ generation plants under the same conditions was 23.86%. The pollen iodine staining rate of wild-type rice 9522 was 89.75%, and the pollen iodine staining rate of homozygous mutant 9522³⁸⁷⁴⁰⁻⁵ T₃ generation plants under the same conditions was 0 (as shown in Table 2 and FIGS. 9A-9D). This indicated that the pollen fertility of the RMS1 mutant material homozygous mutant 9522³⁸⁷⁴⁰⁻⁵ was significantly lower than that of wild-type rice under short light conditions, regardless of the temperature, which further confirmed that the short light conditions were decisive for pollen fertility in RMS1 mutants. At the same time, low temperature also promoted the decrease of pollen fertility in RMS1 mutants under short light conditions.

TABLE 2
Statistics of I₂-KI staining results of pollen grains of RMS1
homozygous mutant 9522³⁸⁷⁴⁰⁻⁵T₃ and wild-type rice variety
9522 under different light conditions

| | | Total | Number of | |
|---|---|---|---|---|
| | | number of | non-stained | |
| | | pollen | pollen | Staining |
| Treatment | Treatment | grains | grains | rate |
| conditions | materials | (grain) | (grain) | (%) |
| Short-day high | 9522 | 2202 | 123 | 94.41 |
| temperature | Homozygous | 570 | 434 | 23.86** |
| treatment | mutant | | | |
| | 9522³⁸⁷⁴⁰⁻⁵ | | | |
| Short-day low | 9522 | 2079 | 213 | 89.75 |
| temperature | Homozygous | 59 | 59 | 0** |
| treatment | mutant | | | |
| | 9522³⁸⁷⁴⁰⁻⁵ | | | |

Note:
**P < 0.01.

(2) The Effect of Temperature on the Pollen Fertility of RMS1 Mutant Rice Under Natural Environment

Under natural conditions in Lingshui, Hainan (18° 51′23″N, 110° 5′6″E, 201912-202004), the RMS1 mutant materials homozygous mutant 9522³⁸⁷⁴⁰⁻⁵ T₄ generation plants and wild-type rice 9522 were planted in two batches. The first batch of materials was sown on Dec. 3, 2019, and its booting period was from Feb. 10, 2020 to Mar. 5, 2020. During this period, the average daily temperature in Lingshui was 22.2° C. The second batch of materials was sown on Dec. 13, 2019, and its booting period was from Feb. 20, 2020 to Mar. 15, 2020. During this period, the daily average temperature in Lingshui was 23.78° C. The difference of average daily temperature between the two batches at booting stage was 1.58° C. After heading, the mature anthers of the homozygous mutant 9522³⁸⁷⁴⁰⁻⁵ T₄ generation plants and the wild-type rice 9522 plants sown in different batches were collected for pollen iodine staining. Three individual plants were randomly selected from each group, and one field of view was taken from each individual plant for statistics.

The results showed that the pollen microscopic staining rate of the first batch of homozygous mutant 9522³⁸⁷⁴⁰⁻⁵ T₄ generation plants was 0%, and the pollen microscopic staining rate of the same batch of wild-type 9522 was 94.87%. while the pollen microscopic staining rate of the homozygous mutant 9522³⁸⁷⁴⁰⁻⁵ T₄ generation plants was 10.97%, and the pollen microscopic staining rate of the same batch of wild-type 9522 was 92.19% (as shown in Table 3). The results indicated that the pollen iodine staining rate of the mutant materials of the same line and sown in batches at the same site was changed, indicating that the temperature difference between different sowing periods could lead to the change of pollen fertility.

TABLE 3
Statistics of pollen grain I₂-KI staining results of RMS1
homozygous mutant 9522³⁸⁷⁴⁰⁻⁵ T₄ plants and wild-type rice
variety 9522 sown in different sowing batches

| | | Total | Number of | |
|---|---|---|---|---|
| | | number of | non-stained | |
| Sowing | Treatment | pollen grains | pollen grains | Staining |
| batches | materials | (grain) | (grain) | rate (%) |
| First | 9522 | 78 | 4 | 94.87 |
| batch | Homozygous | 38 | 38 | 0 |
| | mutant | | | |
| | 9522³⁸⁷⁴⁰⁻⁵ | | | |
| Second | 9522 | 141 | 11 | 92.19 |
| batch | Homozygous | 82 | 73 | 10.97 |
| | mutant | | | |
| | 9522³⁸⁷⁴⁰⁻⁵ | | | |

In conclusion, the pollen of the RMS1 mutant material was sensitive to the length of light, specifically, the pollen of the RMS1 mutant material was fertile under long-day (long light) conditions, The pollen fertility of the short-day (short light) RMS1 mutant material was significantly reduced, and low temperature can promote the complete abortion of mutant RMS1 pollen, which enhanced the pollen sterility of mutant RMS1. Therefore, RMS1 was considered to have the characteristics of anti-photosensitivity and was a photosensitive fertility-related gene, and photosensitive male sterile rice can be obtained by knocking out this gene.

3. Changes in Pollen Fertility of RMS1 Mutant Rice Materials Planted in Different Natural Ecological Regions

In order to further verify the photosensitivity of RMS1, planting experiments in different natural ecological regions were designed. The RMS1 mutant material homozygous mutant 9522³⁸⁷⁴⁰⁻⁵ T₄ generation plants and wild-type rice 9522 were grown under natural conditions in Lingshui of Hainan, Baoshan of Yunnan, Changsha of Hunan, Fuyang of Hangzhou, and Wuhan and Xiangyang of Hubei. After heading, the mature anthers of the homozygous mutant 9522³⁸⁷⁴⁰⁻⁵ T₄ generation plants and wild-type rice 9522 plants grown in different regions were collected for pollen iodine staining. Three individual plants were randomly selected from each group, and one field of view was taken from each individual plant for statistics.

The results showed that (FIG. 10) the pollen fertility of the RMS1 mutant material showed an increasing trend from south to north. Under the natural conditions of Lingshui, Hainan, the pollen of the RMS1 mutant showed complete sterility (the dyeability rate was 0%). Under natural conditions in Xiangyang, Hubei, the pollen fertility of mutant RMS1 was restored (the contamination rate reached 82.35%). It was further indicated that the gene RMS1 was regulated by light to affect pollen fertility, and also showed that the RMS1 mutant material had the potential to be used as a two-line sterile line. Under the natural conditions of Lingshui, Hainan, it can be used as a sterile line to carry out hybrid seed production; under the natural conditions of Xiangyang, Hubei, it can be used as a reproductive line to produce the seeds of the sterile line.

### INDUSTRIAL APPLICATION

The invention utilized CRISPR/Cas9 technology to edit the rice RMS1 gene at a fixed-point, knocked out the rice RMS1 gene through frame shift mutation, inactivated the protein RSM1, and obtained a new generation of rice varieties with photosensitive male sterility (RMS1 gene knockout rice). Compared with wild-type rice, the obtained RMS1 gene knockout rice showed no significant difference in the vegetative growth stage, but the pollen fertility changed with the duration of light. Under short light conditions (the light duration was 10.5 hours, the temperature in the light period was 30° C., the light intensity was 30000 Lx, and the temperature in the dark period was 24° C.), the anthers of the RMS1 gene knockout rice were white and shriveled, and the number of pollen grains decreased significantly. Compared with wild-type rice, pollen iodine staining showed that it contained a large number of sterile pollen grains, and the fertility was significantly reduced; the RMS1 gene knockout rice was exposed to long light conditions (the light duration was 13.5 hours, the temperature in the light period was 30° C., the light intensity was 30000 Lx, and the temperature in the dark period was 24° C.), the anthers of RMS1 knockout rice were bright yellow, plump in shape, and normal in the number of pollen grains, which was consistent with the fertility of wild-type rice, and the fertility was restored compared with mutants under short light conditions. In order to further explore the relationship between pollen fertility and temperature of RMS1 mutant materials, different temperatures were set on the basis of short light conditions. The results showed that the pollen fertility of the RMS1 mutant was significantly lower than that of the wild-type rice under short light conditions, regardless of the temperature, in terms of pollen quantity and dye rate, which further confirmed that the short light conditions had a decisive effect on the pollen fertility of the RMS1 mutant. At the same time, under short light conditions, low temperature also promoted the decrease of pollen fertility of RMS1 mutant and enhanced the pollen sterility of mutant RMS1. This indicated that the RMS1 gene knockout rice was photosensitive male sterile rice (photosensitive male nuclear sterile rice). The photosensitive male sterile rice can be used as a female parent to combine with a dominant variety to produce hybrid seeds. The photosensitive male sterile rice not only provides a new sterile line material for two-line hybrid rice breeding, but also lays a theoretical foundation for hybrid breeding of other gramineous crops.

1-13. (canceled)

14. A method for preparing photosensitive male sterile rice, comprising the following step of: reducing the abundance of protein RMS1 in target rice, reducing the activity of protein RMS1 in target rice or reducing the content of protein RMS1 in target rice, to obtain the photosensitive male sterile rice;
wherein the protein RMS1 is the following A1) or A2):
A1), the amino acid sequence of which is as shown in SEQ ID NO. 1 in the sequence listing;
A2), a homologous protein having more than 98% identity with A1) and is derived from rice.

15. The method according to claim 14, wherein the step of reducing the abundance of protein RMS1 in the target rice, reducing the activity of the protein RMS1 in the target rice or reducing the content of the protein RMS1 in the target rice is implemented by inhibiting the expression of the encoding gene of the protein RMS1 in the target rice or knocking out the encoding gene of the protein RMS1 in the target rice.

16. The method according to claim 15, wherein the step of inhibiting the expression of the encoding gene of the protein RMS1 in the target rice or knocking out the encoding gene of the protein RMS1 in the target rice is implemented by using a CRISPR/Cas9 system.

17. The method according to claim 16, wherein the CRISPR/Cas9 system comprises a vector expressing sgRNA, and the target sequence of the sgRNA is as shown in SEQ ID NO. 15 in the sequence listing.

18. The method according to claim 14, wherein the encoding gene of the protein RMS1 is any one of the following b1)-b4):
b1) a DNA molecule as shown in SEQ ID NO. 2 in the sequence listing;
b2) a DNA molecule as shown in SEQ ID NO. 3 in the sequence listing;
b3) a DNA molecule having 75% or more identity with the nucleotide sequence defined in b1) or b2) and encoding the protein RMS1 of claim 14;
b4) a DNA molecule hybridizing under strict conditions to the nucleotide sequence defined in b1) or b2) and encoding the protein RMS1 of claim 14.

19. Any of the following substances:
(1) an sgRNA or a recombinant plasmid expressing the sgRNA;
wherein the target sequence of the sgRNA is as shown in SEQ ID NO. 15 in the sequence listing;
(2) P1, protein RMS1, which is the following A11) or A12):
A11), the amino acid sequence of which is as shown in SEQ ID NO. 1 in the sequence listing;
A12), a homologous protein having more than 98% identity with A11) and is derived from rice;
(3) P2, protein RMS1-4, which is the following A21) or A22):
A21), the amino acid sequence of which is as shown in SEQ ID NO. 6 in the sequence listing;
A22), a homologous protein having more than 98% identity with A21) and is derived from rice;
(4) P3, protein RMS1-5, which is the following A31) or A32):
A31), the amino acid sequence of which is as shown in SEQ ID NO. 9 in the sequence listing;
A32), a homologous protein having more than 98% identity with A31) and is derived from rice;
(5) P4, protein RMS1-11, which is the following A41) or A42):
A41), the amino acid sequence of which is as shown in SEQ ID NO. 12 in the sequence listing;
A42), a homologous protein having more than 98% identity with A41) and is derived from rice;
(6) Q1, a nucleic acid molecule encoding the protein RMS1 of P1;
(7) Q2, a nucleic acid molecule encoding the protein RMS1-4 of P2;
(8) Q3, a nucleic acid molecule encoding the protein RMS1-5 of P3;
(9) Q4, A nucleic acid molecule encoding the protein RMS1-11 of P4.

20. The substances according to claim 19, wherein:
the nucleic acid molecule encoding the protein RMS1 of P1 is any one of the following b11)-b14):
b11), a DNA molecule shown in SEQ ID NO. 2 in the sequence listing;
b12), a DNA molecule shown in SEQ ID NO. 3 in the sequence listing;
b13), a DNA molecule having 75% or more identity with the nucleotide sequence defined in b11) or b12) and encoding the protein RMS1 of P1;
b14), a DNA molecule that hybridizes to the nucleotide sequence defined in b11) or b12) under strict conditions and encodes the protein RMS1 of P1;
the nucleic acid molecule encoding the protein RMS1-4 of P2 is any one of the following b21)-b24):
b21), a DNA molecule shown in SEQ ID NO. 7 in the sequence listing;
b22), a DNA molecule shown in SEQ ID NO. 8 in the sequence listing;
b23), a DNA molecule having 75% or more identity with the nucleotide sequence defined in b21) or b22) and encoding the protein RMS1-4 of P2;
b24), a DNA molecule that hybridizes to the nucleotide sequence defined in b21) or b22) under strict conditions and encodes the protein RMS1-4 of P2;
the nucleic acid molecule encoding the protein RMS1-5 of P3 is any one of the following b31)-b34):
b31), a DNA molecule shown in SEQ ID NO. 10 in the sequence listing;
b32), a DNA molecule shown in SEQ ID NO. 11 in the sequence listing;
b33), a DNA molecule having 75% or more identity with the nucleotide sequence defined in b31) or b32) and encoding the protein RMS1-5 of P3;
b34), a DNA molecule that hybridizes to the nucleotide sequence defined in b31) or b32) under strict conditions and encodes the protein RMS1-5 of P3;
the nucleic acid molecule encoding the protein RMS1-11 of P4 is any one of the following b41)-b44):
b41), a DNA molecule shown in SEQ ID NO. 13 in the sequence listing;
b42), a DNA molecule shown in SEQ ID NO. 14 in the sequence listing;
b43), a DNA molecule having 75% or more identity with the nucleotide sequence defined in b41) or b42) and encoding the protein RMS1-11 of P4;
b44), a DNA molecule that hybridizes to the nucleotide sequence defined in b41) or b42) under strict conditions and encodes the protein RMS1-11 of P4.

21. Any of the following methods:
(1) a method for rice breeding by using the sgRNA or the recombinant plasmid of claim 19;
(2) a method for preparing transgenic rice, comprising introducing the encoding gene of the sgRNA according to claim 19 and the encoding gene of a Cas9 protein into recipient rice to obtain photosensitive male sterile rice;
(3) a method for regulating rice photoperiod-sensitive fertility by using the protein of claim 19;
(4) a method for cultivating photosensitive male sterile rice by using the protein of claim 19;
(5) a method for cultivating photosensitive male sterile rice by using the protein of claim 19 as a target;
(6) a method for regulating rice photoperiod-sensitive fertility by using the nucleic acid molecule of claim 19;
(7) a method for cultivating photosensitive male sterile rice by using the nucleic acid molecule of claim 19;
(8) a method for cultivating photosensitive male sterile rice by using the nucleic acid molecule of claim 19 as a target.

22. Any of the following methods:
(1) a method for regulating rice photoperiod-sensitive fertility by using the nucleic acid molecule of claim 20;
(2) a method for cultivating photosensitive male sterile rice by using the nucleic acid molecule of claim 20; or
(3) a method for cultivating photosensitive male sterile rice by using the nucleic acid molecule of claim 20 as a target.
